(19) United States
(12) Patent Application Publication
(10) Pub. No.: US 2023/0148931 A1
(43) Pub. Date: May 18, 2023

(54) IMPLANTABLE MEDICAL DEVICES INCLUDING TRANSSEPTAL LEAD

(71) Applicant: Medtronic, Inc., Minneapolis, MN (US)

(72) Inventors: Amy E. Thompson-Nauman, Ham Lake, MN (US); William Eastman, Maple Grove, MN (US); Zhongping Yang, Woodbury, MN (US)

(21) Appl. No.: 17/978,284

(22) Filed: Nov. 1, 2022

Related U.S. Application Data
(60) Provisional application No. 63/280,335, filed on Nov. 17, 2021.

(51) Int. Cl. A61B 5/29; A61B 5/0538; A61B 5/02; A61N 1/37; A61B 5/361; A61B 5/0215

(52) U.S. Cl. CPC A61B 5/29; A61B 5/0538; A61B 5/02028; A61N 1/3706; A61B 5/361; A61B 5/02158

(57) ABSTRACT

Implantable medical devices including a transseptal lead are described herein. The transseptal lead may be positioned or placed through the interatrial septum from the right atrium to the left atrium of a patient's heart and further through the mitral valve. The transseptal lead may include at least one left atrial electrode and at least one left ventricular electrode for sensing, among other things, left atrial and left ventricular electrograms, left atrial and left ventricular impedances, and cross mitral valve impedance.

### RELATED APPLICATIONS

This application claims the benefit of U.S. Provisional Application Ser. No. 63/280,335, filed Nov. 17, 2021, the entire contents of each of which are incorporated herein by reference.

The disclosure herein relates to implantable medical devices for use in sensing cardiac activity in two or more chambers of the patient's heart using a transseptal lead, and methods associated therewith.

Implantable medical devices (IMDs), such as implantable pacemakers, cardioverters, defibrillators, or pacemaker-cardioverter-defibrillators, provide therapeutic electrical stimulation to the heart. IMDs may provide pacing to address bradycardia, or pacing or shocks in order to terminate tachyarrhythmia, such as tachycardia or fibrillation. In some cases, the medical device may sense intrinsic depolarizations of the heart, detect arrhythmia based on the intrinsic depolarizations (or absence thereof), and control delivery of electrical stimulation to the heart if arrhythmia is detected based on the intrinsic depolarizations.

IMDs may also provide cardiac resynchronization therapy (CRT), which is a form of pacing. CRT involves the delivery of pacing to the left ventricle, or both the left and right ventricles. The timing and location of the delivery of pacing pulses to the ventricle(s) may be selected to improve the coordination and efficiency of ventricular contraction.

### SUMMARY

The illustrative implantable medical devices (IMDs) and methods described herein may be configured to sense cardiac activity in two or more chambers of the patient's heart, and perform cardiac therapy to the patient's heart. In some embodiments, the IMDs may include a transseptal lead positionable through the interatrial septum, or atrial septal wall, from the right atrium to the left atrium of a patient's heart to position at least one left atrial electrode in the left atrium and across or through the mitral valve of the patient's heart to position at least one left ventricular electrode in the left ventricle. Further, for example, the IMDs may include at least one right atrial electrode positioned on or coupled to the transseptal lead or another lead such as right lead to position the right atrial electrode in the right atrium of the patient's heart. Still further, for example, a right lead of the IMDs may be positionable through the tricuspid valve and include at least one right ventricular electrode to be positioned in the right ventricle of the patient's heart.

The IMDs may utilize one or more of the at least one left ventricular electrode, the at least one right ventricular electrode, the at least one left atrial electrode, and the at least one right atrial electrode to monitor, sense, or measure electrograms (EGMs) and impedance valves from and across one or more of the left ventricle, right ventricle, left atrium, and right atrium. For example, the IMDs may monitor, sense, or measure left ventricular EGMs, right ventricular EGMs, left atrial EGMs, right atrial EGMs, left ventricular impedance values, right ventricular impedance values, left atrial impedance values, right atrial impedance values, and cross valve impedances such as cross mitral valve impedance and cross tricuspid valve impedance. Such monitoring, sensing, or measuring of values may then be utilized to identify one or more cardiac conditions such as, e.g., indicators of valve disease (e.g., such stenosis, regurgitation, etc.), filling abnormalities, ejection abnormalities, atrial fibrillation, etc. Furthermore, such monitoring, sensing, or measuring of values may then be utilized to determine one or more pacing timings or intervals such as atrioventricular delay or timing interval, intraventricular delay or timing interval, etc.

The IMDs may further include one or more pressure sensors positionable on one or both of a transseptal lead and right ventricular lead to position the one or more pressure sensors in one or more of the left ventricle, right ventricle, left atrium, and right atrium so as to provide pressure data or values from one or more the left ventricle, right ventricle, left atrium, and right atrium. One illustrative IMD may include two pressure sensors; one pressure sensor positionable in the right ventricle and one pressure sensor positionable in the left ventricle. The pressure data or values without or in conjunction with data sensed by the electrodes (such as EGMs) may be used to provide or generate pressure-volume loop data. The pressure-volume loop data may then be used to determine one or more cardiac conditions such as, e.g., hypertrophy, cardiomyopathy, diastolic heart failure, pulmonary congestion, edema, improving or worsening cardiac muscular function, improving or worsening contractility, etc.

One illustrative implantable medical device (IMD) may include a transseptal lead extending from a proximal end to a distal end and comprising at least one left atrial electrode and at least one left ventricular electrode, the transseptal lead positionable through the interatrial septum from the right atrium to the left atrium of a patient's heart to position the at least one left atrial electrode in the left atrium and further through the mitral valve to position the at least one left ventricular electrode in the left ventricle. The IMD may further include a controller comprising one or more processors operably coupled to the transseptal lead and configured to monitor one or more of a cross mitral valve impedance between the at least one left atrial electrode and the at least one left ventricular electrode, a left atrial impedance using the at least one left atrial electrode, and a left ventricular impedance using the at least one left ventricular electrode and determine one or more indicators of mitral valve disease based on one or more of the cross mitral valve impedance, the left atrial impedance, and the left ventricular impedance.

One illustrative method may include monitoring one or more of a cross mitral valve impedance between at least one left atrial electrode positioned in the left atrium and at least one left ventricular electrode positioned int eh left ventricle, a left atrial impedance using the at least one left atrial electrode, and a left ventricular impedance using the at least one left ventricular electrode and determining one or more indicators of mitral valve disease based on one or more of the cross mitral valve impedance, the left atrial impedance, and the left ventricular impedance. The method may further include positioning a transseptal lead through the interatrial septum from the right atrium to the left atrium of a patient's heart to position the at least one left atrial electrode in the left atrium and further through the mitral valve to position the at least one left ventricular electrode in the left ventricle.

One illustrative IMD may include a transseptal lead extending from a proximal end to a distal end and comprising at least one right atrial electrode and at least one left atrial electrode, the transseptal lead positionable through the interatrial septum from the right atrium to the left atrium of a patient's heart to position the at least one right atrial electrode in the right atrium and further through the mitral valve to position the at least one left atrial electrode in the left atrium. The IMD may further include a controller comprising one or more processors operably coupled to the transseptal lead and configured to monitor a right atrial electrogram using the at least one right atrial electrode, monitor a left atrial electrogram using the at least one left atrial electrode, determine right atrium to left atrium electrical activation based on the right and left atrial electrograms, and identify one or more cardiac conditions based on the determined right atrium to left atrium electrical activation.

One illustrative method may include monitoring a right atrial electrogram using at least one right atrial electrode positioned in the right atrium, monitoring a left atrial electrogram using at least one left atrial electrode positioned in the left atrium, determining right atrium to left atrium electrical activation based on the right and left atrial electrograms, and identifying one or more cardiac conditions based on the determined right atrium to left atrium electrical activation. The method may further include positioning a transseptal lead through the interatrial septum from the right atrium to the left atrium of a patient's heart to position the at least one right atrial electrode in the right atrium and further through the mitral valve to position the at least one left atrial electrode in the left atrium.

One illustrative IMD may include a transseptal lead extending from a proximal end to a distal end and comprising at least one left atrial electrode and at least one left ventricular electrode, the transseptal lead positionable through the interatrial septum from the right atrium to the left atrium of a patient's heart to position the at least one left atrial electrode in the left atrium and further through the mitral valve to position the at least one left ventricular electrode in the left ventricle. The IMD may further include a right lead extending from a proximal end to a distal end and comprising at least one right ventricular electrode, the right lead positionable through the right atrium into the right ventricle of the patient's heart to position the at least one right ventricular electrode in the right ventricle, wherein at least one of the transseptal lead and the right lead further comprises at least one right atrial electrode positionable in the right atrium. The IMD may further include a controller comprising one or more processors operably coupled to the transseptal and right leads and configured to monitor one or more of a cross mitral valve impedance between the at least one left atrial electrode and the at least one left ventricular electrode and a cross tricuspid valve impedance between the at least one right atrial electrode and the at least one right ventricular electrode and determine one or more indicators of valve disease based on one or more of the cross mitral valve impedance and the cross tricuspid valve impedance.

One illustrative method may include monitoring one or more of a cross mitral valve impedance between at least one left atrial electrode positioned in the left atrium and at least one left ventricular electrode positioned in the left ventricle and a cross tricuspid valve impedance between at least one right atrial electrode positioned in the right atrium and at least one right ventricular electrode positioned in the right ventricle and determining one or more indicators of valve disease based on one or more of the cross mitral valve impedance and the cross tricuspid valve impedance. The method may further include positioning a transseptal lead through the interatrial septum from the right atrium to the left atrium of a patient's heart to position the at least one left atrial electrode in the left atrium and further through the mitral valve to position the at least one left ventricular electrode in the left ventricle and positioning a right lead through the right atrium into the right ventricle of the patient's heart to position the at least one right ventricular electrode in the right ventricle, wherein at least one of the transseptal lead and the right lead further comprises at least one right atrial electrode positionable in the right atrium.

The above summary is not intended to describe each embodiment or every implementation of the present disclosure. A more complete understanding will become apparent and appreciated by referring to the following detailed description and claims taken in conjunction with the accompanying drawings.

### BRIEF DESCRIPTION OF THE DRAWINGS

FIG. 1 is a diagram of an illustrative implantable medical device (IMD) including a transseptal lead.

FIG. 2 is a diagram of another illustrative IMD including a transseptal lead.

FIG. 3 is a diagram of another illustrative IMD including a transseptal lead and a right lead.

FIG. 4 is a diagram of another illustrative IMD including a transseptal lead and a right lead, each having pressure sensors.

FIG. 5 is a block diagram of the illustrative IMDs of FIGS. 1-4.

FIG. 6 is a flow diagram of a general illustrative method using the IMDs described herein with respect to FIGS. 1-5.

FIG. 7 is a flow diagram of an illustrative method described herein using the IMDs described herein with respect to FIGS. 1-5.

FIG. 8 is a flow diagram of an illustrative method described herein using the IMDs described herein with respect to FIGS. 1-5.

### DETAILED DESCRIPTION OF ILLUSTRATIVE EMBODIMENTS

In the following detailed description of illustrative embodiments, reference is made to the accompanying figures of the drawing which form a part hereof, and in which are shown, by way of illustration, specific embodiments which may be practiced. It is to be understood that other embodiments may be utilized and structural changes may be made without departing from (e.g., still falling within) the scope of the disclosure presented hereby.

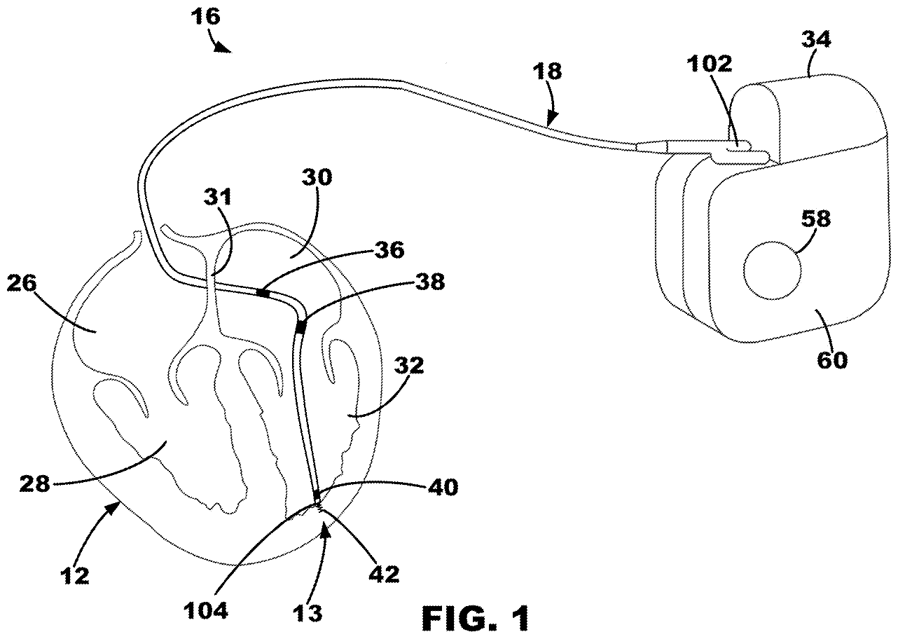

Illustrative devices and methods shall be described with reference to FIGS. 1-8. It will be apparent to one skilled in the art that elements or processes from one embodiment may be used in combination with elements or processes of the other embodiments, and that the possible embodiments of such devices and methods using combinations of features set forth herein is not limited to the specific embodiments shown in the Figures and/or described herein. Further, it will be recognized that the embodiments described herein may include many elements that are not necessarily shown to scale. Still further, it will be recognized that timing of the processes and the size and shape of various elements herein may be modified but still fall within the scope of the present disclosure, although certain timings, one or more shapes and/or sizes, or types of elements, may be advantageous over others.

The illustrative implantable medical devices (IMDs) described herein may be described as including a transseptal lead, which may also be referred to as a left ventricular endocardial lead implant. The transseptal lead may utilize an implant procedure where the transseptal lead passes through the atrial septum, or interatrial septal wall, into the left atrial chamber, across the mitral valve, and into the left ventricular chamber. Once implanted, the transseptal lead may be used to measure or sense, among other things, left atrial electrical-mechanical functioning and left ventricular electrical-mechanical functioning as will be further described herein. In one or more embodiments, the illustrative transseptal lead may include, carry, or have, multiple sensing electrodes, and in some embodiments, pressure sensors, which can be positioned strategically to sense or measure, among other things, left atrium to right atrium timing sequences and activation patterns, atrium to ventricle timing sequences and activation patterns (either right or left heart), cross-valve impedance (one or both of tricuspid cross-valve impedance or mitral cross-valve impedance), pressure-volume loops and all related data (either right or left heart), and other measures of cardiac function. Illustrative IMDs are further described herein with reference to FIGS. 1-5.

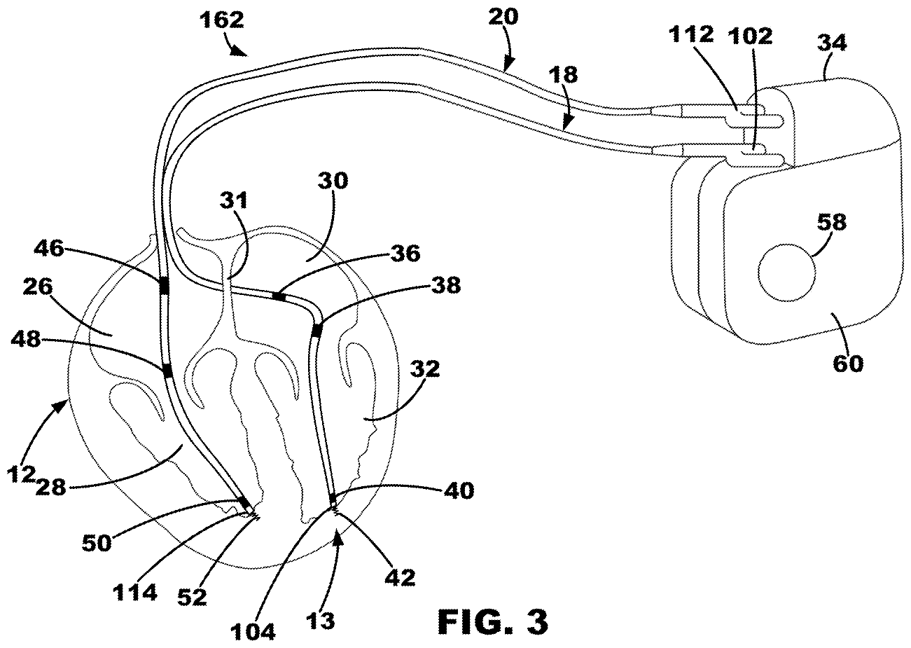

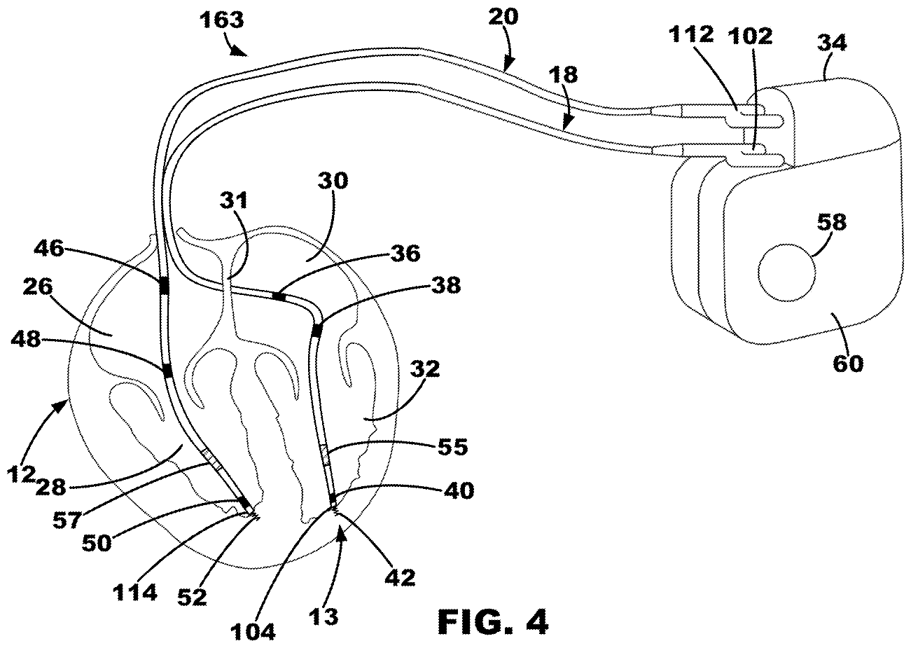

An illustrative IMD 16 that includes, among other things, a transseptal lead 18 and may be used to monitor (e.g., sense, measure, etc.) electrical and mechanical functionality of a patient's heart 12 as well as deliver cardiac pacing therapy thereto is depicted in FIG. 1. The heart 12 may, but not necessarily, be a human heart. The IMD 16 may be, e.g., an implantable pacemaker, cardioverter, and/or defibrillator, that delivers, or provides, electrical signals (e.g., paces, etc.) to and/or senses electrical signals from the patient's heart 12 via electrodes coupled to, at least, transseptal lead 18. Although the IMD 16 of FIG. 1 only includes a single lead, i.e., transseptal lead 18, it is to be understood that the IMD 16 may include more than single lead such as, at least, depicted in FIGS. 3-4. For example, the IMD 16 may include two or more leads, three or more leads etc.

The transseptal lead 18 extends into the patient's heart 12 to sense electrical activity of the heart 12 and/or to deliver electrical stimulation to the heart 12. In the example shown in FIG. 1, the transseptal lead 18 extends through one or more veins (not shown), the superior vena cava (not shown), into the right atrium 26, through the interatrial septal wall or septum 31 into the left atrium 30, and into the left ventricle 32 through the mitral valve. In at least one embodiment, the transseptal lead 18 is placed, or positioned, using a steerable guide catheter acting as a delivery platform across the interatrial septum. Illustrative delivery systems that may be used with the system, devices, apparatus, methods, and processes described herein may be found in U.S. Pat. No. 9,072,872 entitled “Telescoping Catheter Delivery System for Left Heart Endocardial Device Placement” and issued on Jul. 7, 2015, which is incorporated herein by reference in its entirety. Although the transseptal lead 18 as depicted and described herein extends across the mitral valve into the left ventricle, it is to be understood that the transseptal lead 18 may not extend beyond the left atrium, and in such embodiment, may provide one or more electrodes or other sensors in the right and left atria only (which, e.g., may be used to determine right-to-left atrial activation and provide atrial fibrillation monitoring).

More specifically, the transseptal lead 18 may be described as extending from a proximal end 102 to a distal end 104. The proximal end 102 may be operably and physically connected to a connector block 34 of a housing 60 of the IMD 16 as described further herein. The distal end 104 of the transseptal lead 18 may be positioned, or is positionable, in the chamber of the left ventricle 32. In this embodiment, the distal end 104 of the transseptal lead 18 is positioned proximate (e.g., adjacent, in contact with, coupled to, implanted in, etc.) the apex 13 of the left ventricle 32 of the patient's heart 12. In other embodiments, the distal end 104 of the transseptal lead 18 may be positioned proximate (e.g., adjacent, in contact with, coupled to, implanted in, etc.) the apical lateral or mid anterolateral regions of the free wall of the left ventricle 32 of the patient's heart 12. Further in other embodiments, the distal end 104 of the transseptal lead 18 may be positioned proximate (e.g., adjacent, in contact with, coupled to, implanted in, etc.) ventricular septum of the left ventricle 32 of the patient's heart 12. Still, further in other embodiments, the distal end 104 of the transseptal lead 18 may be positioned inferior to the mitral valve within the left ventricle 32 of the patient's heart 12.

The IMD 16 may sense, among other things, electrical signals attendant to the depolarization and repolarization of the heart 12 via electrodes coupled to at least the transseptal lead 18. In some examples, the IMD 16 provides one or both of cardiac electrical sensing and pacing therapy (e.g., pacing pulses) to the heart 12 based on such cardiac electrical sensing of the heart 12. The IMD 16 may be operable to adjust one or more parameters associated with the pacing therapy such as, e.g., A-V delay, V-V delay, and other various timings, pulse wide, amplitude, voltage, burst length, etc. Further, the IMD 16 may be operable to use various electrode configurations to deliver pacing therapy, which may be unipolar, bipolar, quadripoloar, or further multipolar. For example, a multipolar transseptal lead 18 may include several electrodes that can be used for one or both of sensing electrical cardiac activity and delivering pacing therapy. Hence, a multipolar transseptal lead 18 by itself or in conjunction with additional leads may provide, or offer, multiple electrical vectors to pace from. A pacing vector may include at least one cathode, which may be at least one electrode located on at least one lead, and at least one anode, which may be at least one electrode located on at least one lead (e.g., the same lead, or a different lead) and/or on the housing, casing, or can, 60 of the IMD 16. The IMD 16 may also provide defibrillation therapy and/or cardioversion therapy via electrodes located on the transseptal lead 18. Further, the IMD 16 may detect arrhythmia of the heart 12, such as fibrillation of the atria 26, 30 and/or ventricles 28, 32, and deliver defibrillation therapy to the heart 12 in the form of electrical pulses. In some examples, IMD 16 may be programmed to deliver a progression of therapies, e.g., pulses with increasing energy levels, until a fibrillation of the heart 12 ceases or is stopped.

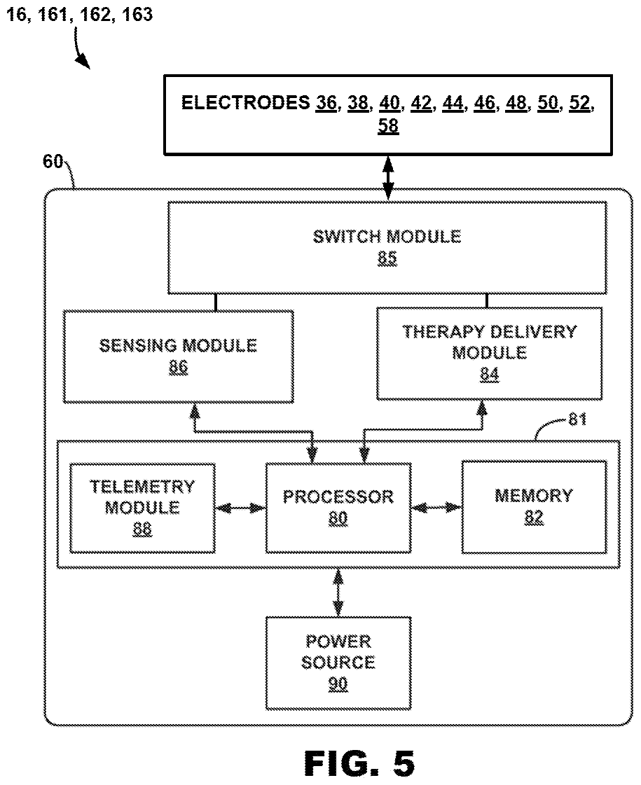

The transseptal lead 18 may be electrically coupled to a therapy delivery module (e.g., for delivery of pacing therapy), a sensing module (e.g., for sensing one or more signals from one or more electrodes), and/or any other modules of the IMD 16 via the connector block 34 as will be further described in FIG. 5. In some examples, the proximal end 102 of the transseptal lead 18 may include electrical contacts that electrically couple to respective electrical contacts within the connector block 34 of the IMD 16. In addition, in some examples, the transseptal lead 18 may be mechanically coupled to the connector block 34 with the aid of set screws, connection pins, or another suitable mechanical coupling mechanism.

The transseptal lead 18 may include an elongated insulative lead body, which may carry a number of conductors (e.g., concentric coiled conductors, straight conductors, etc.) separated from one another by insulation (e.g., tubular insulative sheaths). In the illustrated example, one set of left ventricular electrodes 40, 42 are located proximate to the distal end 104 of the transseptal lead 18 and another set of left atrial electrodes 36, 38 are located proximally from the distal end 104 of the transseptal lead 18. The left ventricular electrodes 40, 42 may be positioned, or coupled, to the transseptal lead 18 at a location to be positioned proximate to (e.g., adjacent, in contact with, coupled to, implanted in, etc.) a target region (e.g., the apex as shown in this embodiment) of the left ventricle 32 when the transseptal lead 18 is implanted so as to one or both of sense cardiac electrical activity and deliver cardiac therapy to such target region in the left ventricle.

Similarly, the left atrial electrodes 36, 38 may be positioned, or coupled, to the transseptal lead 18 at a location to be positioned proximate to (e.g., adjacent, in contact with, coupled to, implanted in, etc.) a target region (e.g., above the mitral valve, in the atrial blood pool, etc. as shown in this embodiment) of the left atrium 30 when the transseptal lead 18 is implanted so as to one or both of sense cardiac electrical activity and deliver cardiac therapy to such target region in the left atrium. Additionally, it is to be understood that the transseptal lead 18 may be configured so as to position the left atrial electrodes 36, 68 proximate the left atrial free wall and atrial septum as well as the atrial appendage.

In this embodiment, the electrodes 36, 38, 40 may take the form of ring electrodes, and the electrode 42 may take the form of helix tip electrode (which may be extendable and mounted retractably within the insulative body of the transseptal lead 18). In another embodiment, the electrode 42 may also take the form of ring electrode or tip electrode. Each of the electrodes 36, 38, 40, 42 may be electrically coupled to a respective one of the conductors (e.g., coiled and/or straight) within the lead body of the transseptal lead 18, and thereby coupled to a respective one of the electrical contacts on the proximal end 102 of the transseptal lead 18.

Additionally, electrodes 36, 38, 40, 42 may have an electrode surface area of about 5.3 mm² to about 5.8 mm². The left atrial electrodes 36, 38 can be spaced apart at variable distances and the left ventricular electrodes 40, 42 spaced apart at variable distances. For example, the left ventricular electrode 42 may be a distance between about 20 millimeters (mm) to about 80 mm away from left ventricular electrode 40. In at least one embodiment, the left ventricular electrode 42 may be a distance of 60 mm from left ventricular electrode 40. In at least one embodiment, the distance between the left ventricular electrode 42 and the left ventricular electrode 40 may be greater than or equal to 20 mm, greater than or equal to 30 mm, greater than or equal to 40 mm, greater than or equal to 50 mm, etc. and/or less than or equal to 80 mm, less than or equal to 70 mm, less than or equal to 60 mm, less than 50 mm, less than 40 mm, etc. For example, the left atrial electrode 36 may be a distance between about 20 mm to about 40 mm away from left atrial electrode 38. In at least one embodiment, the left atrial electrode 36 may be a distance of 30 mm from left atrial electrode 38. In at least one embodiment, the distance between the left atrial electrode 36 and the left atrial electrode 38 may be greater than or equal to 20 mm, greater than or equal to 25 mm, greater than or equal to 30 mm, etc. and/or less than or equal to 40 mm, less than or equal to 35 mm, less than 30 mm, etc.

Additionally, at least one of the left atrial electrodes 36, 38 and at least one of the left ventricular electrodes 40, 42 can be spaced apart at variable distances. For example, at least one of the left ventricular electrodes 40, 42 may be a distance between about 80 mm to about 150 mm away from at least one of the left atrial electrodes 36, 38. In at least one embodiment, at least one of the left ventricular electrodes 40, 42 may be a distance of 125 mm from at least one of the left atrial electrodes 36, 38 (to, e.g., capture cross mitral valve impedance). In at least one embodiment, the distance between at least one of the left ventricular electrodes 40, 42 and at least one of the left atrial electrodes 36, 38 may be greater than or equal to 80 mm, greater than or equal to 90 mm, greater than or equal to 100 mm, greater than or equal to 110 mm, etc. and/or less than or equal to 150 mm, less than or equal to 140 mm, less than or equal to 130 mm, less or equal to 120 mm, etc. Further, the distance between the at least one of the left atrial electrodes 36, 38 and at least one of the left ventricular electrodes 40, 42 may be described in terms of heart anatomy. For example, at least one of the left atrial electrodes 36, 38 may be spaced apart from at least one of the left ventricular electrodes 40, 42 at distance that is approximately the length of a ventricle or atrium, e.g., so as to ensure that at least one of the left atrial electrodes 36, 38 is positioned in the left atrium and at least one of the left ventricular electrodes 40, 42 is positioned in the left ventricle.

As described herein, the electrodes 36, 38, 40, 42 may further be used to sense electrical signals or electrograms (EGMs) (in particular, e.g., morphological waveforms within electrograms (EGMs)) attendant to the depolarization and repolarization of the heart 12. The electrical signals are conducted to the IMD 16 via the conductors within the transseptal lead 18. In some examples, the IMD 16 may also deliver pacing pulses via the electrodes 36, 38, 40, 42 to cause depolarization of cardiac tissue of the patient's heart 12. In some examples, as illustrated in FIG. 1, the IMD 16 includes one or more housing electrodes, such as housing electrode 58, which may be formed integrally with an outer surface of the housing 60 (e.g., a hermetically sealed housing) of the IMD 16 or otherwise coupled to the housing 60. Any of the electrodes 36, 38, 40, 42 may be used for unipolar sensing or pacing in combination with the housing electrode 58. It is generally understood by those skilled in the art that other electrodes can also be selected to define, or be used for, pacing and sensing vectors. Further, any of electrodes 36, 38, 40, 42 when not being used to deliver pacing therapy, may be used to sense electrical activity during pacing therapy.

The illustrative IMD 16 may provide a variety of data regarding the patient's heart that may be useful in determining a patient's cardiac condition and in delivering cardiac therapy to the patient. For example, electrograms (EGM) of the left ventricle and left atrium may be provided by the IMD 16, which may be useful for a variety of things such as, among other things, optimization of pacing timing (e.g., the times at which pacing is delivered, the time periods or intervals between atrial and ventricular activations, etc.). In particular, a left atrial EGM may be monitored using at least one left atrial electrode 36, 38 and a left ventricular EGM may be monitored using at least one left ventricular electrode 40, 42. The left atrial and left ventricular EGMs may be used by the IMD 16 to determine one or more pacing intervals. For example, atrioventricular timing interval extends between a left atrial activation (e.g., either paced left atrial activation or intrinsic left atrial activation) and a left ventricular activation (e.g., either paced left ventricular activation or intrinsic left ventricular activation), and the IMD 16 may utilize the timing (e.g., depolarizations of the left atrium and left ventricle) derived from the left atrial and left ventricular EGMs to determine the time or timing to pace one or both of the left atrium and left ventricle (e.g., a paced activation) using one or more electrodes such as, e.g., electrodes 36, 38, 40, 42.

In at least one embodiment, the left ventricular electrode 42 located at the distal end 104 of the transseptal lead 18 may be used to provide the left ventricular EGM data. In other words, the most distal electrode on the transseptal lead 18 may be used to measure, or sense, cardiac electrical activity of the left ventricle. In at least one embodiment, the left atrial electrode 36 of the transseptal lead 18 located proximal from the other left atrial electrode 38 may be used to provide the left atrial EGM data. In other words, the most proximal electrode on the transseptal lead 18 may be used to measure, or sense, cardiac electrical activity of the left atrium.

Further, for example, impedance data of the left ventricle, left atrium, and therebetween may be provided and used by the IMD 16, which may be useful for a variety of things such as, among other things, determining an approximation of the volume of a heart chamber, determining one or more indicators of mitral valve disease, etc. In particular, for instance, one or more of a cross mitral valve impedance between the at least one left atrial electrode 36, 38 and the at least one left ventricular electrode 40, 42 may be monitored, a left atrial impedance using at least one left atrial electrode 36, 38 may be monitored, and a left ventricular impedance using at least one left ventricular electrode 40, 42 may be monitored. Then, one or more of the cross mitral valve impedance, left atrial impedance, and left ventricular impedance may be used to determine one or more indicators of mitral valve disease such as, e.g., stenosis, regurgitation, etc. Further, one or more of the cross mitral valve impedance, left atrial impedance, and left ventricular impedance may be used to determine one or both of filling abnormalities and ejection abnormalities in one or both of the left atrium and the left ventricle. In one example, the impedance data may first be used to provide chamber volume data over time, which may be further analyzed to determine filling and/or ejection abnormalities and stenosis and/or regurgitation information for the mitral valve. In one example, a surrogate of the forward blood flow rate may be detected by using the at least one left atrial electrode 36, 38 and at least one left ventricular electrode 40, 42 and a surrogate of the back flow rate may be detected by the left atrial electrodes 36, 38, simultaneously.

In at least one embodiment, the left ventricular electrode 40 near the distal end 104 of the transseptal lead 18 may be used to provide the left ventricular impedance data. In other words, the second most distal electrode on the transseptal lead 18 may be used to measure, or sense, electrical impedance of the left ventricle. In at least one embodiment, the left atrial electrode 38 of the transseptal lead 18 located distal from the other left atrial electrode 36 may be used to provide the left atrial impedance data. In other words, the second most proximal electrode on the transseptal lead 18 may be used to measure, or sense, electrical impedance of the left atrium.

As described in further detail with reference to FIG. 5, the housing 60 may enclose a therapy delivery module that may include a stimulation generator for generating cardiac pacing pulses and defibrillation or cardioversion shocks, as well as a sensing module for monitoring the electrical signals of the patient's heart (e.g., the patient's heart rhythm). The transseptal lead 18 may also include elongated electrodes, which may take the form of a coil, which the IMD 16 may use to deliver defibrillation shocks to the heart 12 when used in conjunction with the housing electrode 58. Such elongated electrodes may also be used to deliver cardioversion pulses to the heart 12. Further, the elongated electrodes may be fabricated from any suitable electrically conductive material, such as, but not limited to, platinum, platinum alloy, and/or other materials known to be usable in implantable defibrillation electrodes.

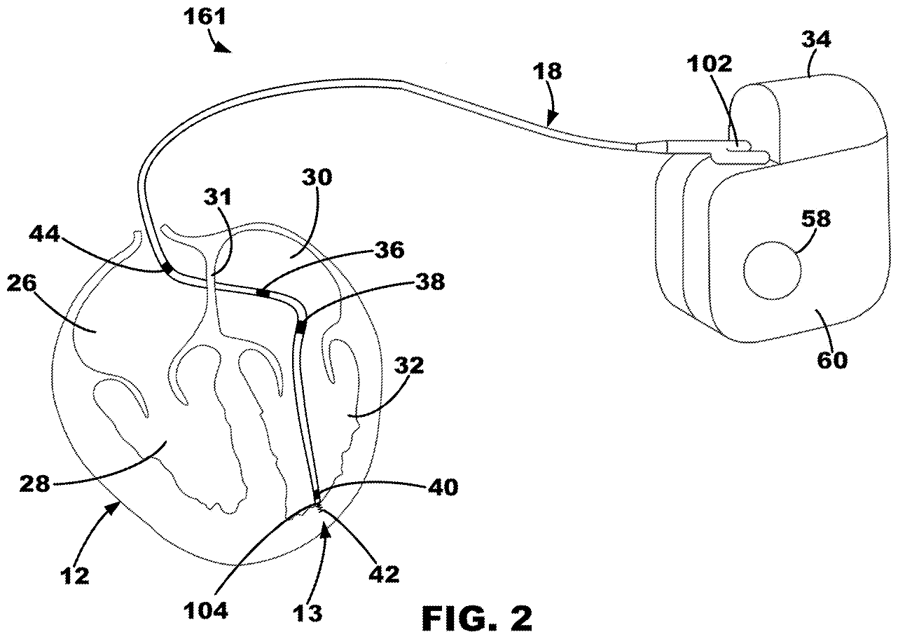

The configuration of the illustrative IMD 16 illustrated in FIG. 1 is merely one example. In other examples such as described herein with respect to FIGS. 2-4, the IMDs may include more electrodes on the transseptal lead 18, additional leads such as a right lead, and more sensors such as pressure sensors carried on or part of one or more leads. Further, in other examples of the illustrative IMDs that provide electrical stimulation therapy to the heart 12, such IMDs may include any suitable number of leads, and each of the leads may extend to any location within or proximate to the heart 12.

Another illustrative IMD 161 that includes, among other things, a transseptal lead 18 and may be used to monitor (e.g., sense, measure, etc.) electrical and mechanical functionality of a patient's heart 12 as well as deliver cardiac pacing therapy thereto is depicted in FIG. 2. The IMD 161 of FIG. 2 is substantially similar to the IMD 16 depicted in FIG. 1, and thus, the parts or portions of the IMD 161 that are substantially similar to the IMD 16 are not described further herein. One difference between the IMD 16 and the IMD 161 is that the transseptal lead 18 of the IMD 161 further includes a right atrial electrode 44.

The right atrial electrode 44 may be positioned, or coupled, to the transseptal lead 18 at a location to be positioned proximate to (e.g., adjacent, in contact with, coupled to, implanted in, etc.) a target region (e.g., above the tricuspid valve, in the atrial blood pool, etc. as shown in this embodiment) of the right atrium 26 when the transseptal lead 18 is implanted so as to one or both of sense cardiac electrical activity and deliver cardiac therapy to such target region in the right atrium 26. The right atrial electrode 44 may be substantially similar to the electrodes 36, 38, 40 described herein. Additionally, although a single right atrial electrode 44 is included in this embodiment, it is to be understood that more than one right atrial electrode may be coupled to (e.g., carried on, positioned on, part of, etc.) the transseptal lead 18 such as, e.g., two or more right atrial electrodes spaced apart on the transseptal lead 18 so as to be positioned in the right atrium 26 when the transseptal lead 18 is implanted in the heart 12. Additionally, it is to be understood that the transseptal lead 18 may be configured so as to position the right atrial electrode 44 as well as any additional right atrial electrodes proximate the right atrial free wall or atrial septum.

The illustrative IMD 161 may provide the same variety of data as the IMD 16 as well as additional data regarding the patient's heart that may be useful in determining a patient's cardiac condition and in delivering cardiac therapy to the patient. For example, electrograms (EGMs) of the left atrium and right atrium may be provided by the IMD 161, which may be useful for a variety of things such as, among other things, optimization of pacing timing (e.g., the times at which pacing is delivered, the time periods or intervals between right atrial and left atrial activations, etc.). In particular, a right atrial EGM may be monitored using at least one right atrial electrode 44 and a left atrial EGM may be monitored using at least one left atrial electrode 36, 38. The left atrial and right atrial EGMs may be used by the IMD 161 to determine one or more pacing intervals. For example, right atrium electrical activation (e.g., either paced or intrinsic) to left atrium electrical activation (e.g., either paced or intrinsic), which may be referred to as the interatrial interval or A-A interval, may be used to configure pacing times (e.g., optimize pacing timings) and identify one or more cardiac conditions based on the determined right atrium to left atrium electrical activation. For example, atrial arrhythmias such as, e.g., atrial fibrillation, may be detected based on right atrial EGMs and left atrial EGMs and/or the interatrial interval. In particular, the atrial electrical propagation may be analyzed and, upon such analysis, may indicate an atrial arrhythmia. Furthermore, the left and right atrial EGMs may also provide various atrial conduction and contraction information.

Another illustrative IMD 162 that includes, among other things, a transseptal lead 18 and may be used to monitor (e.g., sense, measure, etc.) electrical and mechanical functionality of a patient's heart 12 as well as deliver cardiac pacing therapy thereto is depicted in FIG. 3. The IMD 162 of FIG. 3 is substantially similar to the IMD 16 depicted in FIG. 1, and thus, the parts or portions of the IMD 162 that are substantially similar to the IMD 16 are not described further herein. One difference between the IMD 16 and the IMD 162 is that the IMD 162 further includes a right lead 20.

The right lead 20 extends into the patient's heart 12 to sense electrical activity of the heart 12 and/or to deliver electrical stimulation to the heart 12. In the example shown in FIG. 3, the right lead 20 extends through one or more veins (not shown), the superior vena cava (not shown), into the right atrium 26, through the tricuspid valve, and into the right ventricle 28.

More specifically, the right lead 20 may be described as extending from a proximal end 112 to a distal end 114. The proximal end 112 may be operably and physically connected to a connector block 34 of a housing 60 of the IMD 162 as described further herein. The distal end 114 of the right lead 20 may be positioned, or is positionable, in the chamber of the right ventricle 28. In this embodiment, the distal end 114 of the right lead 20 is positioned proximate (e.g., adjacent, in contact with, coupled to, implanted in, etc.) the apex of the patient's right ventricle 28 of the patient's heart 12. In other embodiments, the distal end 114 of the right lead 20 may be positioned proximate (e.g., adjacent, in contact with, coupled to, implanted in, etc.) the apical lateral or mid anterolateral regions of the free wall of the patient's right ventricle 28 of the patient's heart 12. Further in other embodiments, the distal end 114 of the right lead 20 may be positioned proximate (e.g., adjacent, in contact with, coupled to, implanted in, etc.) ventricular septum of the right ventricle 28 of the patient's heart 12. Still, further in other embodiments, the distal end 114 of the right lead 20 may be positioned inferior to the tricuspid valve within the right ventricle 28 of the patient's heart 12.

The right lead 20 may be electrically coupled to a therapy delivery module (e.g., for delivery of pacing therapy), a sensing module (e.g., for sensing one or more signals from one or more electrodes), and/or any other modules of the IMD 162 via the connector block 34 as described in FIG. 5. In some examples, the proximal end 112 of the right lead 20 may include electrical contacts that electrically couple to respective electrical contacts within the connector block 34 of the IMD 162. In addition, in some examples, the right lead 20 may be mechanically coupled to the connector block 34 with the aid of set screws, connection pins, or another suitable mechanical coupling mechanism.

The right lead 20 may include an elongated insulative lead body, which may carry a number of conductors (e.g., concentric coiled conductors, straight conductors, etc.) separated from one another by insulation (e.g., tubular insulative sheaths). In the illustrated example, one set of right ventricular electrodes 50, 52 are located proximate to the distal end 114 of the right lead 20 and another set of right atrial electrodes 46, 48 are located proximally from the distal end 114 of the right lead 20. The right ventricular electrodes 50, 52 may be positioned, or coupled, to the right lead 20 at a location to be positioned proximate to (e.g., adjacent, in contact with, coupled to, implanted in, etc.) a target region (e.g., the apex as shown in this embodiment) of the right ventricle 28 when the right lead 20 is implanted so as to one or both of sense cardiac electrical activity and deliver cardiac therapy to such target region in the right ventricle.

Similarly, the right atrial electrodes 46, 48 may be positioned, or coupled, to the right lead 20 at a location to be positioned proximate to (e.g., adjacent, in contact with, coupled to, implanted in, etc.) a target region (e.g., above the tricuspid valve, in the atrial blood pool, etc. as shown in this embodiment) of the right atrium 26 when the right lead 20 is implanted so as to one or both of sense cardiac electrical activity and deliver cardiac therapy to such target region in the left atrium. The electrodes 46, 48, 50, 52 may be substantially similar (besides positioning and implant location) as the electrodes 36, 38, 40, 42, and thus, not further described herein. Additionally, although two right atrial electrodes 46, 48 and two right ventricular electrodes 50, 52 are included in this embodiment, it is to be understood that a single right atrial electrode or more than two right atrial electrodes and a single right ventricular electrode or more than two right ventricular electrodes may be coupled to (e.g., carried on, positioned on, part of, etc.) the right lead 20.

Additionally, electrodes 46, 48, 50, 52 may have an electrode surface area of about 5.3 mm² to about 5.8 mm². The right atrial electrodes 46, 48 can be spaced apart at variable distances and the right ventricular electrodes 50, 52 spaced apart at variable distances. For example, the right ventricular electrode 52 may be a distance between about 20 mm to about 80 mm away from right ventricular electrode 50. In at least one embodiment, the right ventricular electrode 52 may be a distance of 60 mm from right ventricular electrode 50. In at least one embodiment, the distance between the right ventricular electrode 52 and the right ventricular electrode 50 may be greater than or equal to 20 mm, greater than or equal to 30 mm, greater than or equal to 40 mm, greater than or equal to 50 mm, etc. and/or less than or equal to 80 mm, less than or equal to 70 mm, less than or equal to 60 mm, less than 50 mm, less than 40 mm, etc. For example, the right atrial electrode 46 may be a distance between about 20 mm to about 40 mm away from right atrial electrode 48. In at least one embodiment, the right atrial electrode 46 may be a distance of 30 mm from right atrial electrode 48. In at least one embodiment, the distance between the right atrial electrode 46 and the right atrial electrode 48 may be greater than or equal to 20 mm, greater than or equal to 25 mm, greater than or equal to 30 mm, etc. and/or less than or equal to 40 mm, less than or equal to 35 mm, less than 30 mm, etc.

Additionally, at least one of the right atrial electrodes 46, 48 and at least one of the right ventricular electrodes 50, 52 can be spaced apart at variable distances. For example, at least one of the right ventricular electrodes 50, 52 may be a distance between about 80 mm to about 150 mm away from at least one of the right atrial electrodes 46, 48. In at least one embodiment, at least one of the right ventricular electrodes 50, 52 may be a distance of 125 mm from at least one of the right atrial electrodes 46, 48 (to, e.g., capture cross tricuspid valve impedance). In at least one embodiment, the distance between at least one of the right ventricular electrodes 50, 52 and at least one of the right atrial electrodes 46, 48 may be greater than or equal to 80 mm, greater than or equal to 90 mm, greater than or equal to 100 mm, greater than or equal to 110 mm, etc. and/or less than or equal to 150 mm, less than or equal to 140 mm, less than or equal to 130 mm, etc.

The illustrative IMD 162 may provide the same variety of data as the IMDs 16, 161 as well as additional useful data regarding the patient's heart that may be useful in determining a patient's cardiac condition and in delivering cardiac therapy to the patient. For example, electrograms (EGMs) of the right ventricle and right atrium may be provided by the IMD 162, which may be useful for a variety of things such as, among other things, optimization of pacing timing (e.g., the times at which pacing is delivered, the time periods or intervals between atrial and ventricular activations, etc.). In particular, a right atrial EGM may be monitored using at least one right atrial electrode 46, 48 and a right ventricular EGM may be monitored using at least one right ventricular electrode 50, 52. The right atrial and right ventricular EGMs may be used by the IMD 162 to determine one or more pacing intervals. For example, atrioventricular timing interval extends between an atrial (e.g., right atrial, left atrial, or the earliest or latest of either) activation (e.g., either paced or intrinsic) and a ventricular (e.g., right ventricular, left ventricular, or the earliest or latest of either) activation (e.g., either paced or intrinsic), and the IMD 16 may utilize the timing (e.g., depolarizations of the right atrium, right ventricle, left atrium, and left ventricle) from the right atrial, right ventricular, left atrial, and left ventricular EGMs to determine the time to pace one or more of the right atrium, right ventricle, left atrium, and left ventricle (e.g., a paced activation) using one or more electrodes such as, e.g., electrodes 36, 38, 40, 42, 46, 48, 50, 52. In at least one embodiment, an atrioventricular timing interval between left atrial activation and left ventricular activation may be determined based on one or more of the left atrial and left ventricular EGMs. In at least one embodiment, an intraventricular timing interval between left ventricular activation and right ventricular activation may be determined based on the right ventricular and left ventricular EGMs.

Additionally, such EGMs measured from each chamber of the patient's heart 12 may be used to determine electrical activation timing, or propagation of electrical depolarization, of the patient's heart 12, and thus, may be used to determine dyssynchrony, or electrical heterogeneity, of the patient's heart 12. Then, the IMD 162 may identify one or more cardiac conditions based on the determined dyssynchrony, or electrical heterogeneity, of the patient's heart 12.

In at least one embodiment, the right ventricular electrode 52 located at the distal end 114 of the right lead 20 may be used to provide the right ventricular EGM data. In other words, the most distal electrode on the right lead 20 may be used to measure, or sense, cardiac electrical activity of the right ventricle. In at least one embodiment, the right atrial electrode 46 of the right lead 20 located proximal from the other right atrial electrode 48 may be used to provide the right atrial EGM data. In other words, the most proximal electrode on the right lead 20 may be used to measure, or sense, cardiac electrical activity of the right atrium.

Further, for example, impedance data of the left ventricle, left atrium, right ventricle, right atrium, and therebetween may be provided by the IMD 162, which may be useful for a variety of things such as, among other things, determining an approximation of the volume of a heart chamber, determining one or more indicators of mitral valve disease, determining one or more indicators of tricuspid valve disease, etc. In particular, for instance, one or more of a cross tricuspid valve impedance between the at least one right atrial electrode 46, 48 and the at least one right ventricular electrode 50, 52 may be monitored, a cross mitral valve impedance between the at least one left atrial electrode 36, 38 and the at least one left ventricular electrode 40, 42 may be monitored, a right atrial impedance using at least one right atrial electrode 46, 48 may be monitored, and a right ventricular impedance using at least one right ventricular electrode 50, 52 may be monitored. a left atrial impedance using at least one left atrial electrode 36, 38 may be monitored, and a left ventricular impedance using at least one left ventricular electrode 40, 42 may be monitored.

Then, one or more of the cross-tricuspid valve impedance, cross mitral valve impedance, right atrial impedance, right ventricular impedance, left atrial impedance, and left ventricular impedance may be used to determine one or more indicators of mitral and/or tricuspid valve disease such as, e.g., stenosis, regurgitation, etc. Further, one or more of the cross-tricuspid valve impedance, cross mitral valve impedance, right atrial impedance, right ventricular impedance, left atrial impedance, and left ventricular impedance may be used to determine one or both of filling abnormalities and ejection abnormalities in one or more of the right atrium, right ventricle, left atrium, and the left ventricle. The impedance data may first be used to provide chamber volume data over time, which may be further analyzed to determine filling and/or ejection abnormalities and stenosis and/or regurgitation information for the tricuspid valve and/or mitral valve. In one example, a surrogate of the forward blood flow rate through the left side of the heart may be detected by using the at least one left atrial electrode 36, 38 and at least one left ventricular electrode 40, 42 and a surrogate of the back flow rate through the left side of the heart may be detected by the left atrial electrodes 36, 38, simultaneously. In one example, a surrogate of the forward blood flow rate through the right side of the heart may be detected by using the at least one right atrial electrode 46, 48 and at least one right ventricular electrode 50, 52 and a surrogate of the back flow rate through the right side of the heart may be detected by the right atrial electrodes 46, 48, simultaneously.

In at least one embodiment, the right ventricular electrode 50 near the distal end 114 of the right lead 20 may be used to provide the right ventricular impedance data. In other words, the second most distal electrode on the right lead 20 may be used to measure, or sense, electrical impedance of the right ventricle. In at least one embodiment, the right atrial electrode 48 of the right lead 20 located distal from the other right atrial electrode 46 may be used to provide the right atrial impedance data. In other words, the second most proximal electrode on the right lead 20 may be used to measure, or sense, electrical impedance of the right atrium.

Another illustrative IMD 163 that includes, among other things, a transseptal lead 18 and may be used to monitor (e.g., sense, measure, etc.) electrical and mechanical functionality of a patient's heart 12 as well as deliver cardiac pacing therapy thereto is depicted in FIG. 4. The IMD 163 of FIG. 4 is substantially similar to the IMD 162 depicted in FIG. 3, and thus, the parts or portions of the IMD 163 that are substantially similar to the IMD 162 are not described further herein. One difference between the IMD 162 and the IMD 163 is that each of the transseptal lead 18 and the right lead 20 of the IMD 163 further includes pressure sensors 55, 57, respectively.

The left ventricular pressure senor 55 may be positioned, or coupled, to the transseptal lead 18 at a location to be positioned proximate to (e.g., adjacent, in contact with, coupled to, implanted in, etc.) a target region (e.g., in the ventricular blood pool, below the mitral valve, etc. as shown in this embodiment) of the left ventricle 32 when the transseptal lead 18 is implanted so as to sense left ventricular pressure (i.e., pressure within the chamber of the left ventricle). The left ventricular pressure sensed by the left ventricular pressure sensor 55 may be transmitted as an electrical signal via one or more conductors of the transseptal lead 18 to the housing 60 of the IMD 163, where the controller and other circuitry of the IMD 163 may process such signal to determine the pressure of the left ventricle.

Similarly, the right ventricular pressure sensor 57 may be positioned, or coupled, to the right lead 20 at a location to be positioned proximate to (e.g., adjacent, in contact with, coupled to, implanted in, etc.) a target region (e.g., in the ventricular blood pool, below the tricuspid valve, etc. as shown in this embodiment) of the right ventricle 28 when the right lead 20 is implanted so as to sense right ventricular pressure (i.e., pressure within the chamber of the right ventricle). The right ventricular pressure sensed by the right ventricular pressure sensor 57 may be transmitted as an electrical signal via one or more conductors of the right lead 20 to the housing 60 of the IMD 163, where the controller and other circuitry of the IMD 163 may process such signal to determine the pressure of the right ventricle.

Additionally, although a single left ventricular pressure sensor 55 and a single right ventricular pressure sensor 57 is included in this embodiment, it is to be understood that more than one left ventricular pressure sensor and more than one right ventricular pressure sensor may be coupled to (e.g., carried on, positioned on, part of, etc.) the transseptal lead 18 and the right lead 20.

The illustrative IMD 163 may provide the same variety of data as the IMDs 16, 161, 162 as well as additional data regarding the patient's heart that may be useful in determining a patient's cardiac condition and in delivering cardiac therapy to the patient. For example, right ventricular pressure and left ventricular pressure may be provided by the IMD 163, which may be useful for a variety of things such as, among other things, monitoring cardiac health or disease of a patient. In particular, right ventricular pressure may be monitored using the right ventricular pressure sensor 57 and left ventricular pressure may be monitored using the left ventricular pressure sensor 55. The right and left ventricular pressures, or pressure data, may be used in conjunction with the impedance and EGM data described herein to provide complete pressure-volume loop data. The pressure-volume loop data may include left and right ventricular pressures and left and right ventricle chamber volumes or sizes (e.g., determined from the impedance data). The pressure-volume loop data may then be used to determine information indicative of hypertrophy and cardiomyopathy (e.g., complete cardiac cycle information), analyze contractility data, determine information indicative of diastolic heart failure, determine information indicative of pulmonary congestion or edema, and information indicative of cardiac muscular function.

In at least one embodiment, the left ventricular pressure sensor 55 may be positioned in the left ventricle 32 when the transseptal lead 18 is implanted. The left ventricular pressure sensor 55 may be positioned proximally from the left ventricular electrodes 40, 42. In at least one embodiment, the right ventricular pressure sensor 57 may be positioned in the right ventricle 28 when the right lead 20 is implanted. The right ventricular pressure sensor 57 may be positioned proximally from the right ventricular electrodes 50, 52.

FIG. 5 is a functional block diagram of one illustrative configuration of the IMDs 16, 161, 162, 163. As shown, the IMDs 16, 161, 162, 163 may include a control module 81, a therapy delivery module 84 (e.g., which may include a stimulation generator), a sensing module 86, and a power source 90 within the housing 60.

The control module, or apparatus, 81 may include a processor or processing circuitry 80, memory 82, and a telemetry module, or apparatus, 88. The memory 82 may include computer-readable instructions that, when executed, e.g., by the processor 80, cause the IMDs 16, 161, 162, 163 and/or the control module 81 to perform various functions attributed to the IMDs 16, 161, 162, 163 and/or the control module 81 described herein. Further, the memory 82 may include any volatile, non-volatile, magnetic, optical, and/or electrical media, such as a random-access memory (RAM), read-only memory (ROM), non-volatile RAM (NVRAM), electrically-erasable programmable ROM (EEPROM), flash memory, and/or any other digital media.

The processor 80 of the control module 81 may include any one or more of a microprocessor, a controller, a digital signal processor (DSP), an application specific integrated circuit (ASIC), a field-programmable gate array (FPGA), and/or equivalent discrete or integrated logic circuitry. In some examples, the processor 80 may include multiple components, such as any combination of one or more microprocessors, one or more controllers, one or more DSPs, one or more ASICs, and/or one or more FPGAs, as well as other discrete or integrated logic circuitry. The functions attributed to the processor 80 herein may be embodied as software, firmware, hardware, or any combination thereof.

The control module 81 may control the therapy delivery module 84 to deliver therapy (e.g., electrical stimulation therapy such as pacing) to the heart 12 according to a selected one or more therapy programs, which may be stored in the memory 82. More specifically, the control module 81 (e.g., the processor 80) may control various parameters of the electrical stimulus delivered by the therapy delivery module 84 such as, e.g., A-V delays, V-V delays, pacing pulses with the amplitudes, pulse widths, frequency, or electrode polarities, etc., which may be specified by one or more selected therapy programs (e.g., A-V and/or V-V delay adjustment programs, pacing therapy programs, pacing recovery programs, capture management programs, etc.). As shown, the therapy delivery module 84 is electrically coupled to electrodes 36, 38, 40, 42, 44, 46, 48, 50, 52, 58 e.g., via conductors of the respective lead 18, 20 or, in the case of housing electrode 58, via an electrical conductor disposed within the housing 60. Therapy delivery module 84 may be configured to generate and deliver electrical stimulation therapy such as pacing therapy to the heart 12 using one or more of the electrodes 36, 38, 40, 42, 44, 46, 48, 50, 52, 58.

For example, the therapy delivery module 84 may deliver pacing stimulus (e.g., pacing pulses) via ring electrodes 36, 38, 40, 44, 46, 48, 50, coupled to leads 18, 20 and/or helical tip electrodes 42, 52 of leads 18, 20. Further, for example, the therapy delivery module 84 may deliver defibrillation shocks or cardioversion pulses to heart 12 via at least one of coil electrode and the housing electrode 58. In some examples, therapy delivery module 84 may be configured to deliver pacing, cardioversion, or defibrillation stimulation in the form of electrical pulses. In other examples, therapy delivery module 84 may be configured to deliver one or more of these types of stimulation in the form of other signals, such as sine waves, square waves, and/or other substantially continuous time signals. Further, for example, the therapy delivery module 84 may deliver impedance measure pulses (e.g., small pulses) via ring electrodes 36, 38, 40, 44, 46, 48, 50, coupled to leads 18, 20 and/or helical tip electrodes 42, 52 of leads 18, 20 for use to measure impedance values.

The IMDs 16, 161, 162, 163 may further include a switch module 85 and the control module 81 (e.g., the processor 80) may use the switch module 85 to select, e.g., via a data/address bus, which of the available electrodes are used to deliver therapy such as pacing pulses for pacing therapy, or which of the available electrodes are used for sensing. The switch module 85 may include a switch array, switch matrix, multiplexer, or any other type of switching device suitable to selectively couple the sensing module 86 and/or the therapy delivery module 84 to one or more selected electrodes. More specifically, the therapy delivery module 84 may include a plurality of pacing output circuits. Each pacing output circuit of the plurality of pacing output circuits may be selectively coupled, e.g., using the switch module 85, to one or more of the electrodes 36, 38, 40, 42, 44, 46, 48, 50, 52, 58 (e.g., a pair of electrodes for delivery of therapy to a bipolar or multipolar pacing vector). In other words, each electrode can be selectively coupled to one of the pacing output circuits of the therapy delivery module using the switch module 85.

The sensing module 86 is coupled (e.g., electrically coupled) to sensing apparatus, which may include, among additional sensing apparatus, the electrodes 36, 38, 40, 42, 44, 46, 48, 50, 52, 58 to monitor electrical activity of the heart 12, e.g., electrocardiogram (ECG)/electrogram (EGM) signals, impedance signals or values, etc. The ECG/EGM signals may be used to measure or monitor activation times (e.g., ventricular activations times, etc.), heart rate (HR), heart rate variability (HRV), heart rate turbulence (HRT), deceleration/acceleration capacity, deceleration sequence incidence, T-wave alternans (TWA), P-wave to P-wave intervals (also referred to as the P-P intervals or A-A intervals), R-wave to R-wave intervals (also referred to as the R-R intervals or V-V intervals), P-wave to QRS complex intervals (also referred to as the P-R intervals, A-V intervals, or P-Q intervals), QRS-complex morphology, ST segment (i.e., the segment that connects the QRS complex and the T-wave), T-wave changes, QT intervals, electrical vectors, etc. The impedance signals or data may be used to provide chamber volumes and cross valve information.

The switch module 85 may also be used with the sensing module 86 to select which of the available electrodes are used, or enabled, to, e.g., sense electrical activity of the patient's heart (e.g., one or more electrical vectors of the patient's heart using any combination of the electrodes 36, 38, 40, 42, 44, 46, 48, 50, 52, 58). Likewise, the switch module 85 may also be used with the sensing module 86 to select which of the available electrodes are not to be used (e.g., disabled) to, e.g., sense electrical activity of the patient's heart (e.g., one or more electrical vectors of the patient's heart using any combination of the electrodes 36, 38, 40, 42, 44, 46, 48, 50, 52, 58), etc. In some examples, the control module 81 may select the electrodes that function as sensing electrodes via the switch module within the sensing module 86, e.g., by providing signals via a data/address bus.

In some examples, sensing module 86 includes a channel that includes an amplifier with a relatively wider pass band than the R-wave or P-wave amplifiers. Signals from the selected sensing electrodes may be provided to a multiplexer, and thereafter converted to multi-bit digital signals by an analog-to-digital converter for storage in memory 82, e.g., as an electrogram (EGM). In some examples, the storage of such EGMs in memory 82 may be under the control of a direct memory access circuit.

In some examples, the control module 81 may operate as an interrupt-driven device and may be responsive to interrupts from pacer timing and control module, where the interrupts may correspond to the occurrences of sensed P-waves and R-waves and the generation of cardiac pacing pulses. Any necessary mathematical calculations may be performed by the processor 80 and any updating of the values or intervals controlled by the pacer timing and control module may take place following such interrupts. A portion of memory 82 may be configured as a plurality of recirculating buffers, capable of holding one or more series of measured intervals, which may be analyzed by, e.g., the processor 80 in response to the occurrence of a pace or sense interrupt to determine whether the patient's heart 12 is presently exhibiting atrial or ventricular tachyarrhythmia.

The telemetry module 88 of the control module 81 may include any suitable hardware, firmware, software, or any combination thereof for communicating with another device, such as a programmer. For example, under the control of the processor 80, the telemetry module 88 may receive downlink telemetry from and send uplink telemetry to a programmer with the aid of an antenna, which may be internal and/or external. The processor 80 may provide the data to be unlinked to a programmer and the control signals for the telemetry circuit within the telemetry module 88, e.g., via an address/data bus. In some examples, the telemetry module 88 may provide received data to the processor 80 via a multiplexer.

The various components of the IMDs 16, 161, 162, 163 are further coupled to a power source 90, which may include a rechargeable or non-rechargeable battery. A non-rechargeable battery may be selected to last for several years, while a rechargeable battery may be inductively charged from an external device, e.g., on a daily or weekly basis.

The techniques described in this disclosure, including those attributed to the IMDs 16, 161, 162, 163 and/or various constituent components, may be implemented, at least in part, in hardware, software, firmware, or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, image processing devices, or other devices. The term “module,” “processor,” or “processing circuitry” may generally refer to any of the foregoing logic circuitry, alone or in combination with other logic circuitry, or any other equivalent circuitry.

Such hardware, software, and/or firmware may be implemented within the same device or within separate devices to support the various operations and functions described in this disclosure. In addition, any of the described units, modules, or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed by processing circuitry and/or one or more processors to support one or more aspects of the functionality described in this disclosure.

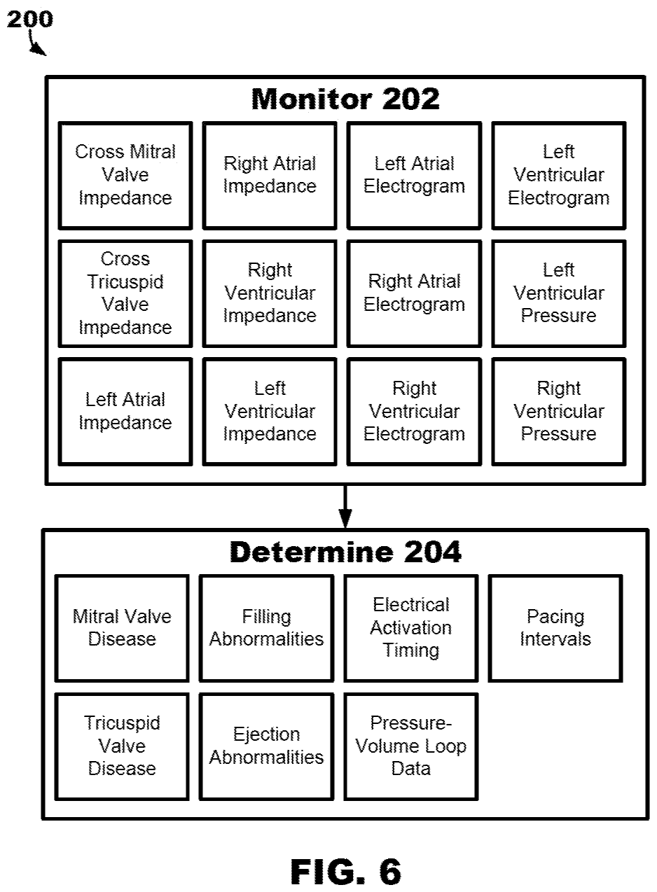

A flow diagram of a general illustrative method 200 using the IMDs described herein with respect to FIGS. 1-5 is shown in FIG. 6. A shown, the method 200 may monitor 202 a plurality of different metrics using the IMDs of FIGS. 1-5. In particular, the method 200 may monitor 202 one or more of cross mitral valve impedance, cross tricuspid valve impedance, left atrial impedance, right atrial impedance, right ventricular impedance, left ventricular impedance, left atrial electrograms, right atrial electrograms, right ventricular electrograms, left ventricular electrograms, left ventricular pressure, and right ventricular pressure. Using one or more of the metrics monitored in process 202, the method 200 may determine one or more results. In other words, the method 200 may determine one or more results based on one or more of the monitored metrics. In particular, the method 200 may determine 204 one or more of mitral valve disease (e.g., indicators thereof), tricuspid valve disease (e.g., indicators thereof), filling abnormalities in the right atrium, right ventricle, left atrium, and left ventricle, ejection abnormalities in the right atrium, right ventricle, left atrium, and left ventricle, electrical activation timing (including right-to-left atrial activation, left-to-right ventricular activation, atria-to-left ventricular activation, etc.), pressure-volume loop data, and pacing intervals. In essence, the IMDs of FIGS. 1-5 may be configured to collect a plurality of different data types, which may then be used to determine a plurality of different cardiac conditions or issues. Further, the determination 204 of one or more conditions or issues may then be used to generate an alert or notification to a patient or care provider using a telemetry module 88 as described herein with reference to FIG. 5. For instance, if the IMDs and methods described herein detect a patient is undergoing or in atrial fibrillation, a notification may be sent to a patient's mobile phone or other device.

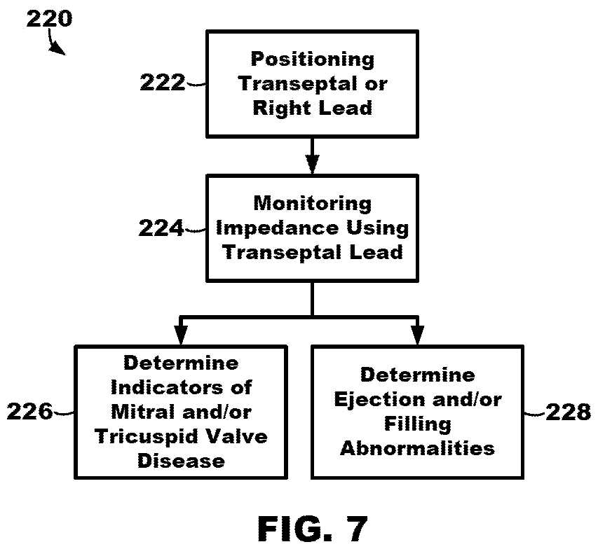

A flow diagram of an illustrative method 220 described herein using the IMDs described herein with respect to FIGS. 1-5 is shown in FIG. 7. The method 220 may include positioning (e.g., implanted) a transseptal lead 222 through the interatrial septum from the right atrium to the left atrium of a patient's heart to position at least one left atrial electrode in the left atrium and further through the mitral valve to position at least one left ventricular electrode in the left ventricle. Various impedances may then be monitored 224 using the transseptal lead. For example, a cross mitral valve impedance may be monitored 224 across the mitral valve using at least one left atrial electrode and at least one left ventricular electrode. Additionally, impedance of the left atrium and the left ventricle may be monitored 224. The transseptal lead may further include one or more right atrial electrodes to be positioned in the right atrium, and thus, right atrial impedance may also be monitored.

The method 220 may include positioning (e.g., implanted) a right lead 222 through the right atrium into the right ventricle of the patient's heart to position at least one right ventricular electrode in the right ventricle and optionally at least one right atrial electrode in the right atrium. Various impedances may then be monitored 224 using the right lead and/or transseptal lead. For example, a cross tricuspid valve impedance may be monitored 224 across the tricuspid valve using at least one right atrial electrode and at least one right ventricular electrode. Additionally, impedance of the right atrium and the right ventricle may be monitored 224.

Using such monitored impedances, the method 220 may determining indicators of mitral or tricuspid valve disease 226 and determining ejection and/or filling abnormalities 228 of the right atrium, left atrium, right ventricle, and left ventricle. For example, the cross mitral valve impedance based be used to determine indicators of mitral valve disease. Further, for example, the cross-tricuspid valve impedance based be used to determine indicators of tricuspid valve disease. Right atrial impedance may be used to determine ejection and/or filling abnormalities of the right atrium. Left atrial impedance may be used to determine ejection and/or filling abnormalities of the left atrium. Right ventricular impedance may be used to determine ejection and/or filling abnormalities of the right ventricle. Left ventricular impedance may be used to determine ejection and/or filling abnormalities of the left ventricle.

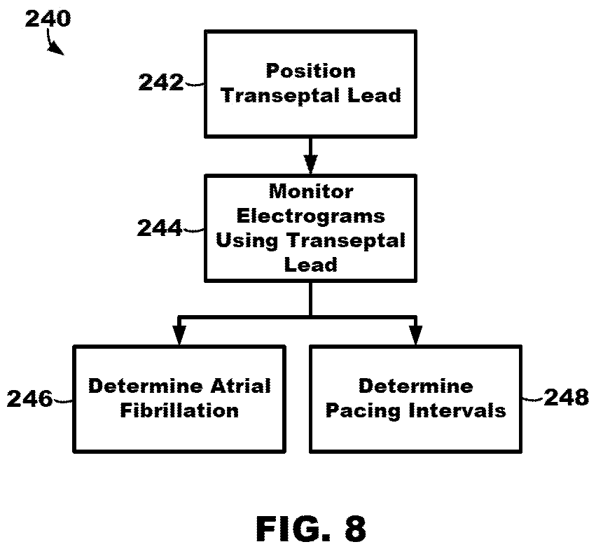

A flow diagram of an illustrative method 240 described herein using the IMDs described herein with respect to FIGS. 1-5 is shown in FIG. 8. The method 240 may include positioning (e.g., implanted) a transseptal lead 242 through the interatrial septum from the right atrium to the left atrium of a patient's heart to position one or more of at least one left atrial electrode in the left atrium and at least one right atrial electrode in the right atrium and further through the mitral valve to position at least one left ventricular electrode in the left ventricle. Various electrograms (EGMs) may then be monitored 244 using the transseptal lead. For example, a right atrial EGM may be monitored 244 using at least one right atrial electrode. Further, for example, a left atrial EGM may be monitored 244 using at least one left atrial electrode. Still further, for example, a left ventricular EGM may be monitored 244 using at least one left ventricular electrode.

The monitored EGMs may then be analyzed to determine atrial fibrillation 246 and to determine various pacing intervals 248. For example, one or more of the right atrial, left atrial, and left ventricular EGMs may be analyzed for electrical activity indicative of atrial fibrillation. For instance, the EGMs may be reviewed to determine whether one or more missing or subdued P-waves and irregular ventricular contractions exist, and in response thereto, it may be determined that the patient is undergoing or in atrial fibrillation. Additionally, for example, an atrioventricular pacing delay may be determined (e.g., determined periodically) by sensing an intrinsic atrioventricular delay using one or both atrial electrodes and a left ventricular electrode and then setting the atrioventricular pacing delay to a percentage or fraction of intrinsic atrioventricular delay.

### ILLUSTRATIVE EMBODIMENTS

Embodiment 1: An implantable medical device (IMD) coupled to a transseptal lead extending from a proximal end to a distal end and comprising at least one left atrial electrode and at least one left ventricular electrode, the transseptal lead positionable through the interatrial septum from the right atrium to the left atrium of a patient's heart to position the at least one left atrial electrode in the left atrium and further through the mitral valve to position the at least one left ventricular electrode in the left ventricle; and
a controller comprising one or more processors operably coupled to the transseptal lead and configured to:
   monitor one or more of a cross mitral valve impedance between the at least one left atrial electrode and the at least one left ventricular electrode, a left atrial impedance using the at least one left atrial electrode, and a left ventricular impedance using the at least one left ventricular electrode; and
   determine one or more indicators of mitral valve disease based on one or more of the cross mitral valve impedance, the left atrial impedance, and the left ventricular impedance.

Embodiment 2: A method comprising:
monitoring one or more of a cross mitral valve impedance between at least one left atrial electrode positioned in the left atrium and at least one left ventricular electrode positioned int eh left ventricle, a left atrial impedance using the at least one left atrial electrode, and a left ventricular impedance using the at least one left ventricular electrode; and determining one or more indicators of mitral valve disease based on one or more of the cross mitral valve impedance, the left atrial impedance, and the left ventricular impedance.

Embodiment 3: The method of embodiment 2, the method further comprising positioning a transseptal lead through the interatrial septum from the right atrium to the left atrium of a patient's heart to position the at least one left atrial electrode in the left atrium and further through the mitral valve to position the at least one left ventricular electrode in the left ventricle.

Embodiment 4: The IMD or method as in any one of embodiments 1-3, wherein the one or more indicators of mitral valve disease comprises one or more stenosis and regurgitation.

Embodiment 5: The IMD or method as in any one of embodiments 1-4, wherein the controller is further configured to execute or the method further comprises determining filling abnormalities in one or both of the left atrium and the left ventricle based on one or more of the cross mitral valve impedance, the left atrial impedance, and the left ventricular impedance.

Embodiment 6: The IMD or method as in any one of embodiments 1-5, wherein the controller is further configured to execute or the method further comprises determining ejection abnormalities in one or both of the left atrium and the left ventricle based on one or more of the cross mitral valve impedance, the left atrial impedance, and the left ventricular impedance.

Embodiment 7: The IMD or method as in any one of embodiments 1-6, wherein the at least one left atrial electrode comprises a first left atrial electrode to monitor the left atrial impedance and a second left atrial electrode to monitor the cross mitral valve impedance, and
wherein the at least one left ventricular electrode comprises a first left ventricular electrode to monitor the left ventricular impedance and a second left ventricular electrode to monitor the cross mitral valve impedance with the second left atrial electrode.

Embodiment 8: The IMD or method as in any one of embodiments 1-7, wherein the controller is further configured to execute or the method further comprises:
monitoring a left atrial electrogram using the at least one left atrial electrode;
monitoring a left ventricular electrogram using the at least one left ventricular electrode; and
determining one or more pacing intervals based on the left atrial and left ventricular electrograms.

Embodiment 9: The IMD or method of embodiment 8, wherein the one or more pacing intervals comprises an atrioventricular timing interval extending between a left atrial activation and a left ventricular activation.

Embodiment 10: The IMD as in any one of embodiments 1 and 4-9 or the method as in any one of embodiments 3-9, wherein the transseptal lead further comprises at least one right atrial electrode positionable in the right atrium, wherein the controller is further configured to execute or the method further comprises:
monitoring a right atrial electrogram using the at least one right atrial electrode;
monitoring a left atrial electrogram using the at least one left atrial electrode;
determining right atrium to left atrium electrical activation based on the right and left atrial electrograms; and
identifying one or more cardiac conditions based on the determined right atrium to left atrium electrical activation.

Embodiment 11: The IMD as in any one of embodiments 1-8, wherein the at least one left ventricular electrode comprises a coil electrode to be attached proximate the apical region of the left ventricle.

Embodiment 12: An implantable medical device (IMD) comprising:
a transseptal lead extending from a proximal end to a distal end and comprising at least one right atrial electrode and at least one left atrial electrode, the transseptal lead positionable through the interatrial septum from the right atrium to the left atrium of a patient's heart to position the at least one right atrial electrode in the right atrium and further through the mitral valve to position the at least one left atrial electrode in the left atrium; and
a controller comprising one or more processors operably coupled to the transseptal lead and configured to:
   monitor a right atrial electrogram using the at least one right atrial electrode;
   monitor a left atrial electrogram using the at least one left atrial electrode;
   determine right atrium to left atrium electrical activation based on the right and left atrial electrograms; and
   identify one or more cardiac conditions based on the determined right atrium to left atrium electrical activation.

Embodiment 13: A method comprising:
monitoring a right atrial electrogram using at least one right atrial electrode positioned in the right atrium;
monitoring a left atrial electrogram using at least one left atrial electrode positioned in the left atrium;
determining right atrium to left atrium electrical activation based on the right and left atrial electrograms; and
identifying one or more cardiac conditions based on the determined right atrium to left atrium electrical activation.

Embodiment 14: The method of embodiment 13, the method further comprising positioning a transseptal lead through the interatrial septum from the right atrium to the left atrium of a patient's heart to position the at least one right atrial electrode in the right atrium and further through the mitral valve to position the at least one left atrial electrode in the left atrium.

Embodiment 15: The IMD or method as in any one of embodiments 12-14, wherein the one or more cardiac conditions comprises atrial fibrillation.

Embodiment 16: An implantable medical device (IMD) comprising:
a transseptal lead extending from a proximal end to a distal end and comprising at least one left atrial electrode and at least one left ventricular electrode, the transseptal lead positionable through the interatrial septum from the right atrium to the left atrium of a patient's heart to position the at least one left atrial electrode in the left atrium and further through the mitral valve to position the at least one left ventricular electrode in the left ventricle;
   a right lead extending from a proximal end to a distal end and comprising at least one right ventricular electrode, the right lead positionable through the right atrium into the right ventricle of the patient's heart to position the at least one right ventricular electrode in the right ventricle, wherein at least one of the transseptal lead and the right lead further comprises at least one right atrial electrode positionable in the right atrium; and
   a controller comprising one or more processors operably coupled to the transseptal and right leads and configured to:
   monitor one or more of a cross mitral valve impedance between the at least one left atrial electrode and the at least one left ventricular electrode and a cross tricuspid valve impedance between the at least one right atrial electrode and the at least one right ventricular electrode; and
   determine one or more indicators of valve disease based on one or more of the cross mitral valve impedance and the cross tricuspid valve impedance.

Embodiment 17: A method comprising:
monitoring one or more of a cross mitral valve impedance between at least one left atrial electrode positioned in the left atrium and at least one left ventricular electrode positioned in the left ventricle and a cross tricuspid valve impedance between at least one right atrial electrode positioned in the right atrium and at least one right ventricular electrode positioned in the right ventricle; and
determining one or more indicators of valve disease based on one or more of the cross mitral valve impedance and the cross tricuspid valve impedance.

Embodiment 18: The method of embodiment 17, the method further comprising:
positioning a transseptal lead through the interatrial septum from the right atrium to the left atrium of a patient's heart to position the at least one left atrial electrode in the left atrium and further through the mitral valve to position the at least one left ventricular electrode in the left ventricle; and
positioning a right lead through the right atrium into the right ventricle of the patient's heart to position the at least one right ventricular electrode in the right ventricle, wherein at least one of the transseptal lead and the right lead further comprises at least one right atrial electrode positionable in the right atrium.

Embodiment 19: The IMD or method as in any one of embodiments 16-18, wherein the one or more indicators of valve disease comprises one or more stenosis and regurgitation.

Embodiment 20: The IMD or method as in any one of embodiments 16-19, wherein the controller is further configured to execute or the method further comprises:
monitoring one or more of a right atrial impedance using the at least one right atrial electrode, a right ventricular impedance using the at least one right ventricular electrode, a left atrial impedance using the at least one left atrial electrode, and a left ventricular impedance using the at least one left ventricular electrode; and
determining filling abnormalities in one or more of the right atrium, the right ventricle, the left atrium, and the left ventricle based on one or more of the right atrial impedance, the right ventricular impedance, the left atrial impedance, and the left ventricular impedance.

Embodiment 21: The IMD or method as in any one of embodiments 16-20, wherein the controller is further configured to execute or the method further comprises:
monitoring one or more of a right atrial impedance using the at least one right atrial electrode, a right ventricular impedance using the at least one right ventricular electrode, a left atrial impedance using the at least one left atrial electrode, and a left ventricular impedance using the at least one left ventricular electrode; and
determining ejection abnormalities in one or more of the right atrium, the right ventricle, the left atrium, and the left ventricle based on one or more of the right atrial impedance, the right ventricular impedance, the left atrial impedance, and the left ventricular impedance.

Embodiment 22: The IMD or method as in any one of embodiments 16-21,
wherein the at least one right atrial electrode comprises a first right atrial electrode to monitor the right atrial impedance and a second right atrial electrode to monitor the cross-tricuspid valve impedance, and
wherein the at least one right ventricular electrode comprises a first right ventricular electrode to monitor the right ventricular impedance and a second right ventricular electrode to monitor the cross-tricuspid valve impedance with the second right atrial electrode,
wherein the at least one left atrial electrode comprises a first left atrial electrode to monitor the left atrial impedance and a second left atrial electrode to monitor the cross mitral valve impedance, and
wherein the at least one left ventricular electrode comprises a first left ventricular electrode to monitor the left ventricular impedance and a second left ventricular electrode to monitor the cross mitral valve impedance with the second left atrial electrode.

Embodiment 23: The IMD or method as in any one of embodiments 16-22, wherein the controller is further configured to execute or the method further comprises:
monitoring a right atrial electrogram using the at least one right atrial electrode;
monitoring a right ventricular electrogram using the at least one right ventricular electrode;
monitoring a left atrial electrogram using the at least one left atrial electrode;
monitoring a left ventricular electrogram using the at least one left ventricular electrode; and
determining one or more pacing intervals based on the right atrial, right ventricular, left atrial, and left ventricular electrograms.

Embodiment 24: The IMD or method of embodiment 23, wherein the one or more pacing intervals comprises at least one of an atrioventricular timing interval between left atrial activation and left ventricular activation and an intraventricular timing interval between left ventricular activation and right ventricular activation.

Embodiment 25: The IMD or method as in any one of embodiments 16-24, wherein the controller is further configured to execute or the method further comprises:
monitoring a right atrial electrogram using the at least one right atrial electrode;
monitoring a right ventricular electrogram using the at least one right ventricular electrode;
monitoring a left atrial electrogram using the at least one left atrial electrode;
monitoring a left ventricular electrogram using the at least one left ventricular electrode;
determining cardiac electrical activation timing of the patient's heart based on the right atrial, right ventricular, left atrial, and left ventricular electrograms; and
identifying one or more cardiac conditions based on the determined cardiac electrical activation timing of the patient's heart.

Embodiment 26: The IMD or method as in any one of embodiments 16 and 18-25, wherein the transseptal lead further comprises a left ventricular pressure sensor positionable in the left ventricle and the right lead further comprises a right ventricular pressure sensor positionable in the right ventricle, wherein the controller is further configured to monitor right ventricular pressure using the right ventricular pressure sensor and monitor left ventricular pressure using the left ventricular pressure sensor.

Embodiment 27: The IMD or method as in any one of embodiments 16 and 18-25, wherein the transseptal lead further comprises a left ventricular pressure sensor positionable in the left ventricle and the right lead further comprises a right ventricular pressure sensor positionable in the right ventricle, wherein the controller is further configured to execute or the method further comprises:
monitoring right ventricular pressure using the right ventricular pressure sensor and monitor left ventricular pressure using the left ventricular pressure sensor;
monitoring one or more of a right atrial impedance using the at least one right atrial electrode, a right ventricular impedance using the at least one right ventricular electrode, a left atrial impedance using the at least one left atrial electrode, and a left ventricular impedance using the at least one left ventricular electrode; and
determining pressure-volume loop data based on the right and left ventricular pressures, right atrial impedance, right ventricular impedance, left atrial impedance, and left ventricular impedance.

Embodiment 28: The IMD or method of embodiment 27, wherein the pressure-volume loop data comprises information indicative of hypertrophy and cardiomyopathy.

Embodiment 29: The IMD or method as in any one of embodiments 27-28, wherein the pressure-volume loop data comprises contractility data.

Embodiment 30: The IMD or method as in any one of embodiments 27-29, wherein the pressure-volume loop data comprises information indicative of diastolic heart failure.

Embodiment 31: The IMD or method as in any one of embodiments 27-30, wherein the pressure-volume loop data comprises information indicative of pulmonary congestion or edema.

Embodiment 32: The IMD or method as in any one of embodiments 27-30, wherein the pressure-volume loop data comprises information indicative of cardiac muscular function.

This disclosure has been provided with reference to illustrative embodiments and is not meant to be construed in a limiting sense. As described previously, one skilled in the art will recognize that other various illustrative applications may use the techniques as described herein to take advantage of the beneficial characteristics of the devices and methods described herein. Various modifications of the illustrative embodiments, as well as additional embodiments of the disclosure, will be apparent upon reference to this description.

1. An implantable medical device (IMD) comprising:
a controller comprising one or more processors operably coupled to a transseptal lead extending from a proximal end to a distal end and comprising at least one left atrial electrode and at least one left ventricular electrode, the transseptal lead positionable through the interatrial septum from the right atrium to the left atrium of a patient's heart to position the at least one left atrial electrode in the left atrium and further through the mitral valve to position the at least one left ventricular electrode in the left ventricle, the controller configured to:
monitor one or more of a cross mitral valve impedance between the at least one left atrial electrode and the at least one left ventricular electrode, a left atrial impedance using the at least one left atrial electrode, and a left ventricular impedance using the at least one left ventricular electrode; and
determine one or more indicators of mitral valve disease based on one or more of the cross mitral valve impedance, the left atrial impedance, and the left ventricular impedance.

2. The IMD of claim 1, wherein the one or more indicators of mitral valve disease comprises one or more stenosis and regurgitation.

3. The IMD of claim 1, wherein the controller is further configured to determine filling abnormalities in one or both of the left atrium and the left ventricle based on one or more of the cross mitral valve impedance, the left atrial impedance, and the left ventricular impedance.

4. The IMD of claim 1, wherein the controller is further configured to determine ejection abnormalities in one or both of the left atrium and the left ventricle based on one or more of the cross mitral valve impedance, the left atrial impedance, and the left ventricular impedance.

5. The IMD of claim 1, wherein the at least one left atrial electrode comprises a first left atrial electrode and a second left atrial electrode, wherein the controller is configured to monitor the left atrial impedance using the first left atrial electrode and monitor the cross mitral valve impedance using the second left atrial electrode, and
wherein the at least one left ventricular electrode comprises a first left ventricular electrode and a second left ventricle electrode, wherein the controller is configured to monitor the left ventricular impedance using the second left ventricular electrode and monitor the cross mitral valve impedance using the second left ventricle electrode.

6. The IMD of claim 1, wherein the controller is further configured to:
monitor a left atrial electrogram using the at least one left atrial electrode;
monitor a left ventricular electrogram using the at least one left ventricular electrode; and
determine one or more pacing intervals based on the left atrial and left ventricular electrograms.

7. The IMD of claim 6, wherein the one or more pacing intervals comprises an atrioventricular timing interval extending between a left atrial activation and a left ventricular activation.

8. The IMD of claim 1, wherein the transseptal lead further comprises at least one right atrial electrode positionable in the right atrium, wherein the controller is further configured to:
monitor a right atrial electrogram using the at least one right atrial electrode;
monitor a left atrial electrogram using the at least one left atrial electrode;
determine right atrium to left atrium electrical activation based on the right and left atrial electrograms; and
identify one or more cardiac conditions based on the determined right atrium to left atrium electrical activation.

9. An implantable medical device (IMD) comprising:
a transseptal lead extending from a proximal end to a distal end and comprising at least one left atrial electrode and at least one left ventricular electrode, the transseptal lead positionable through the interatrial septum from the right atrium to the left atrium of a patient's heart to position the at least one left atrial electrode in the left atrium and further through the mitral valve to position the at least one left ventricular electrode in the left ventricle;
a right lead extending from a proximal end to a distal end and comprising at least one right ventricular electrode, the right lead positionable through the right atrium into the right ventricle of the patient's heart to position the at least one right ventricular electrode in the right ventricle, wherein at least one of the transseptal lead and the right lead further comprises at least one right atrial electrode positionable in the right atrium; and
a controller comprising one or more processors operably coupled to the transseptal and right leads and configured to:
monitor one or more of a cross mitral valve impedance between the at least one left atrial electrode and the at least one left ventricular electrode and a cross tricuspid valve impedance between the at least one right atrial electrode and the at least one right ventricular electrode; and
determine one or more indicators of valve disease based on one or more of the cross mitral valve impedance and the cross tricuspid valve impedance.

10. The IMD of claim 9, wherein the one or more indicators of valve disease comprises one or more stenosis and regurgitation.

11. The IMD of claim 9, wherein the controller is further configured to:
monitor one or more of a right atrial impedance using the at least one right atrial electrode, a right ventricular impedance using the at least one right ventricular electrode, a left atrial impedance using the at least one left atrial electrode, and a left ventricular impedance using the at least one left ventricular electrode; and
determine filling abnormalities in one or more of the right atrium, the right ventricle, the left atrium, and the left ventricle based on one or more of the right atrial impedance, the right ventricular impedance, the left atrial impedance, and the left ventricular impedance.

12. The IMD of claim 9, wherein the controller is further configured to:
monitor one or more of a right atrial impedance using the at least one right atrial electrode, a right ventricular impedance using the at least one right ventricular electrode, a left atrial impedance using the at least one left atrial electrode, and a left ventricular impedance using the at least one left ventricular electrode; and
determine ejection abnormalities in one or more of the right atrium, the right ventricle, the left atrium, and the left ventricle based on one or more of the right atrial impedance, the right ventricular impedance, the left atrial impedance, and the left ventricular impedance.

13. The IMD of claim 9, wherein the at least one right atrial electrode comprises a first right atrial electrode and a second right atrial electrode, wherein the controller is configured to monitor the right atrial impedance using the first right atrial electrode and monitor the cross tricuspid valve impedance using the second right atrial electrode,
wherein the at least one right ventricular electrode comprises a first right ventricular electrode and a second right ventricle electrode, wherein the controller is configured to monitor the right ventricular impedance using the first right ventricular electrode and monitor the cross tricuspid valve impedance with the second right ventricle,
wherein the at least one left atrial electrode comprises a first left atrial electrode and a second left atrial electrode, wherein the controller is configured to monitor the left atrial impedance using the first left atrial electrode and monitor the cross mitral valve impedance using the second left atrial electrode, and
wherein the at least one left ventricular electrode comprises a first left ventricular electrode and a second left ventricle electrode, wherein the controller is configured to monitor the left ventricular impedance using the second left ventricular electrode and monitor the cross mitral valve impedance using the second left ventricle electrode.

14. The IMD of claim 9, wherein the controller is further configured to:
monitor a right atrial electrogram using the at least one right atrial electrode;
monitor a right ventricular electrogram using the at least one right ventricular electrode;
monitor a left atrial electrogram using the at least one left atrial electrode;
monitor a left ventricular electrogram using the at least one left ventricular electrode; and
determine one or more pacing intervals based on the right atrial, right ventricular, left atrial, and left ventricular electrograms.

15. The IMD of claim 14, wherein the one or more pacing intervals comprises at least one of an atrioventricular timing interval between left atrial activation and left ventricular activation and an intraventricular timing interval between left ventricular activation and right ventricular activation.

16. The IMD of claim 9, wherein the controller is further configured to:
monitor a right atrial electrogram using the at least one right atrial electrode;
monitor a right ventricular electrogram using the at least one right ventricular electrode;
monitor a left atrial electrogram using the at least one left atrial electrode;
monitor a left ventricular electrogram using the at least one left ventricular electrode;
determine cardiac electrical activation timing of the patient's heart based on the right atrial, right ventricular, left atrial, and left ventricular electrograms; and
identify one or more cardiac conditions based on the determined cardiac electrical activation timing of the patient's heart.

17. The IMD of claim 9, wherein the transseptal lead further comprises a left ventricular pressure sensor positionable in the left ventricle and the right lead further comprises a right ventricular pressure sensor positionable in the right ventricle, wherein the controller is further configured to monitor right ventricular pressure using the right ventricular pressure sensor and monitor left ventricular pressure using the left ventricular pressure sensor.

18. The IMD of claim 9, wherein the transseptal lead further comprises a left ventricular pressure sensor positionable in the left ventricle and the right lead further comprises a right ventricular pressure sensor positionable in the right ventricle, wherein the controller is further configured to:
monitor right ventricular pressure using the right ventricular pressure sensor and monitor left ventricular pressure using the left ventricular pressure sensor;
monitor one or more of a right atrial impedance using the at least one right atrial electrode, a right ventricular impedance using the at least one right ventricular electrode, a left atrial impedance using the at least one left atrial electrode, and a left ventricular impedance using the at least one left ventricular electrode; and
determine pressure-volume loop data based on the right and left ventricular pressures, right atrial impedance, right ventricular impedance, left atrial impedance, and left ventricular impedance.

19. The IMD of claim 18, wherein the pressure-volume loop data comprises information indicative of hypertrophy and cardiomyopathy.

20. The IMD of claim 18, wherein the pressure-volume loop data comprises contractility data.

21. The IMD of claim 18, wherein the pressure-volume loop data comprises information indicative of diastolic heart failure.

22. The IMD of claim 18, wherein the pressure-volume loop data comprises information indicative of pulmonary congestion or edema.

23. The IMD of claim 18, wherein the pressure-volume loop data comprises information indicative of cardiac muscular function.

24. An implantable medical device (IMD) comprising:
a transseptal lead extending from a proximal end to a distal end and comprising at least one right atrial electrode and at least one left atrial electrode, the transseptal lead positionable through the interatrial septum from the right atrium to the left atrium of a patient's heart to position the at least one right atrial electrode in the right atrium and further through the mitral valve to position the at least one left atrial electrode in the left atrium; and
a controller comprising one or more processors operably coupled to the transseptal lead and configured to:
monitor a right atrial electrogram using the at least one right atrial electrode;
monitor a left atrial electrogram using the at least one left atrial electrode;
determine right atrium to left atrium electrical activation based on the right and left atrial electrograms; and
identify one or more cardiac conditions based on the determined right atrium to left atrium electrical activation.

25. The IMD of claim 24, wherein the one or more cardiac conditions comprises atrial fibrillation.
